(19) United States
(12) Patent Application Publication
(10) Pub. No.: US 2020/0061102 A1
(43) Pub. Date: Feb. 27, 2020

(54) COMPOSITION FOR PROTECTING RADIATION COMPRISING POLY-GAMMA-GLUTAMATE AND USES THEREOF

(71) Applicant: KOOKMINBIO, CORP., Jeollabuk-do (KR)

(72) Inventors: Moon-Hee SUNG, Seoul (KR); Mi-Sun KWAK, Goyang-si (KR); Yu Jin OH, Busan (KR); Sang-Joon PARK, Yangju-si (KR)

(73) Assignee: KOOKMINBIO, CORP., Jeollabuk-do (KR)

(21) Appl. No.: 16/421,493

(22) Filed: May 24, 2019

(30) Foreign Application Priority Data
Aug. 21, 2018 (KR) 10-2018-0097348

(51) Int. Cl. A61K 31/785; A61P 39/00

(52) U.S. Cl. CPC A61K 31/785; G21F 1/10; A61P 39/00

(57) ABSTRACT

The present invention relates to a composition for shielding radiation comprising biomaterial poly-gamma-glutamic acid, and it is confirmed that the polygamma-glutamic acid polymer exhibits a radiation shielding effect up to 87% at a concentration of 0.33 to 1.65 μM and thus a composition comprising the poly-gamma-glutamic acid is provided as a material for shielding radiation, and a radiation shielding material using the composition and a method of preparing the same are provided.

### CROSS-REFERENCE TO RELATED APPLICATION

This application claims priority to and the benefit of Korean Patent Application No. 10-2018-0097348 filed in the Korean Intellectual Property Office on Aug. 21, 2018, the entire contents of which are incorporated herein by reference.

### BACKGROUND OF THE DISCLOSURE

### 1. Field of the Disclosure

The present invention relates to a composition for shielding radiation comprising a biomaterial poly-gamma-glutamic acid.

### 2. Description of the Related Art

Humans have been very widely exposed by radiation. With the increasing use of nuclear facilities and radiation therapy, interest in radioprotective agents has increased after the Fukushima nuclear accident in Japan in 2011. Typical radioprotective agents include amifostine and glutathione (GSH), but these agents have been reported to have side effects.

Amifostine has been specially approved by the US Food and Drug Administration (FDA) for its use as a radio prophylactic agent and it is known as a drug that can be used clinically for radiation therapy because it has selective protective action against normal tissues and tumor cells as a radioprotective agent which is currently available for clinical use. However, several clinical and animal studies have reported several side effects such as nausea, vomiting, drowsiness, acute hypotension, hypocalcemia, disgust and allergic reactions. (Johnke, Roberta M., Jennifer A. Sattler, and Ron R. Allison, “Radioprotective agents for radiation therapy: future trends.” Future oncology 10.15 (2014): 2345-2357.)

Accordingly, radiation is exposed to nuclear power-related workers, radiographer, researchers, doctors, workers and patients receiving radiotherapy in daily life and therefore, it is necessary to study a material having excellent radiation shielding function while solving the problem of side effects of conventional radioprotective agents.

Poly-γ-glutamic acid (γ-PGA) is a biodegradable, water-soluble, anionic and edible amino acid polymer produced from Bacillus subtilis, GRAS (generally regarded as safe) microorganism isolated from Korean traditional fermented food, Cheonggukjang (fast-fermented bean paste) and it has a wide range of applications as medicines, functional foods and moisturizing cosmetic materials. γ-PGA is a safe ingredient with no toxicity to humans, as a component of Cheonggukjang that our ancestors have eaten for thousands of years.

### PRIOR ART DOCUMENT

### Patent Document

(Patent Document 1) Korean Patent No. 10-1708840 (published on Feb. 22, 2017)

### SUMMARY OF THE DISCLOSURE

It is an object of the present invention to provide a radiation shielding raw material comprising poly-gamma-glutamic acid, a radiation shielding raw material comprising poly-gamma-glutamic acid and a method of preparing a radiation shielding product using poly-gamma-glutamic by confirming an effect of radiation shielding of a biomaterial poly-gamma-glutamic acid.

The present invention provides a composition for shielding radiation comprising poly-gamma-glutamic acid as an active ingredient.

The present invention provides a radiation shielding material comprising poly-gamma-glutamic acid as an active ingredient.

Also, the present invention provides a method of preparing radiation shielding product comprising: mixing the composition for shielding radiation of claim 1 with an object requiring radiation transmission prevention or blocking, or placing the composition on the object, or coating the composition on the object or immobilizing the composition on the object.

### BRIEF DESCRIPTION OF THE DRAWINGS

FIG. 1 shows the results of measuring CT-DNA in which damage was induced by irradiation. When gamma rays were irradiated at a total dose of 3756 Gy at a dose of 62.6 Gy/min, gamma-irradiated DNA was reacted with EtBr and the fluorescence emission spectrum was measured at a wavelength of 624 nm, it was confirmed that the fluorescence intensity was gradually decreased as the irradiation dose was increased as compared with the EtBr-DNA control. FIG. 1a shows the fluorescence intensity of only 60 μM EtBr; FIG. 1b shows the fluorescence intensity of 60 μM CT-DNA bound to EtBr without irradiation with gamma radiation; FIG. 1c shows the fluorescence intensity of 60 μM CT-DNA bound to EtBr at a dose of 626 Gy; FIG. 1d shows the fluorescence intensity of 60 μM CT-DNA bound to EtBr at a dose of 1252 Gy; FIG. 1e shows the fluorescence intensity of 60 μM CT-DNA bound to EtBr at a dose of 1878 Gy; FIG. 1f shows the fluorescence intensity of 60 μM CT-DNA bound to EtBr at a dose of 2504 Gy; FIG. 1g shows the fluorescence intensity of 60 μM CT-DNA bound to EtBr at dose of 3130 Gy; and FIG. 1h is the fluorescence intensity of 60 μM CT-DNA bound to EtBr at 3756 Gy dose.

FIG. 2 shows the results of measuring CT-DNA in which damage was induced by irradiation. When gamma rays were irradiated at a total dose of 3756 Gy at a dose of 62.6 Gy/min, gamma-irradiated DNA was reacted with EtBr and the fluorescence emission spectrum was measured at a wavelength of 624 nm, it was confirmed that the fluorescence intensity was gradually decreased as the irradiation dose was increased as compared with the EtBr-DNA control. The degree of damage of CT-DNA induced by irradiation was expressed as DNA damage rate (%) and the degree of DNA damage induced by radiation was calculated using the formula (I-Iₐ)/(I₀Iₐ). When the fluorescence intensity of EtBr was denoted by Iₐ, the fluorescence intensity of the EtBr-DNA control was denoted by I₀ and the fluorescence intensity of the gamma-irradiated DNA-EtBr was denoted by I, the degree of DNA damage induced by radiation was expressed as DNA damage rate (%). The present invention is a result of confirming that the DNA strand break induced by the gamma ray is almost a linear graph for the irradiation amount of the gamma ray.

FIG. 3 shows a results of protection effect of CT-DNA in which damage is induced by 1252 Gy irradiation at which D₅₀ (radiation dose at which 50% of DNA is damaged) of polygamma-glutamic acid is induced, i.e. the result of showing the radiation shielding rate of polygamma-glutamic acid protecting the gamma-ray irradiated CT-DNA damage and γ-PGA-added DNA was irradiated with 1252 Gy gamma rays and then reacted with EtBr to measure fluorescence emission spectrum at a wavelength of 624 nm. FIG. 3a shows the fluorescence intensity of only 60 μM EtBr; FIG. 3b shows the fluorescence intensity of 60 μM CT-DNA bound to EtBr without irradiation of gamma rays; FIG. 3c shows the fluorescence intensity of 60 μM CT-DNA bound to EtBr at a dose of 1252 Gy of D₅₀ (radiation dose at which 50% of DNA is damaged); FIG. 3d shows the fluorescence intensity of 60 μM CT-DNA bound to EtBr at a dose of 1252 Gy of 1.65 μM D/L glutamate-added D₅₀ (radiation dose at which 50% of DNA is damaged); FIG. 3e shows the fluorescence intensity of 60 μM CT-DNA bound to EtBr at a dose of 1252 Gy of 0.1 wt % γ-PGA-added D₅₀ (radiation dose at which 50% of DNA is damaged); FIG. 3f shows the fluorescence intensity of 60 μM CT-DNA bound to EtBr at a dose of 1252 Gy of 0.2 wt % γ-PGA-added D₅₀ (radiation dose at which 50% of DNA is damaged); FIG. 3g shows the fluorescence intensity of 60 μM CT-DNA bound to EtBr at a dose of 1252 Gy of 0.3 wt % γ-PGA-added D₅₀ (radiation dose at which 50% of DNA is damaged); FIG. 3h shows the fluorescence intensity of 60 μM CT-DNA bound to EtBr at a dose of 1252 Gy of 0.4 wt % γ-PGA-added D₅₀ (radiation dose at which 50% of DNA is damaged); and FIG. 3i shows the fluorescence intensity of 60 μM CT-DNA bound to EtBr at a dose of 1252 Gy of 0.5 wt % γ-PGA-added D₅₀ (radiation dose at which 50% of DNA is damaged).

FIG. 4 shows the protection effect of CT-DNA in which damage is induced by irradiation at a dose of 1252 Gy of D₅₀ (radiation dose at which 50% of DNA is damaged) of γ-PGA as a result of calculating the gamma ray shielding rate (%) with respect to the ratio of the molar concentration of γ-PGA to the molar concentration of CT-DNA by (I-Iₐ)/(I₀Iₐ). As the gamma ray shielding rate (%), the degree of DNA damage induced by radiation was calculated from (I-Iₐ)/(I₀-Iₐ). When the fluorescence intensity of EtBr was denoted by Iₐ, the fluorescence intensity of the EtBr-DNA control was denoted by I₀ and the fluorescence intensity of the gamma-irradiated DNA-EtBr was denoted by I, as a result of the graph of (I-Iₐ)/(I₀-Iₐ) according to the gamma ray irradiation, the DNA damage is gradually protected as the amount of γ-PGA increases, and the gamma ray shielding rate is 87%.

### DETAILED DESCRIPTION OF THE EMBODIMENTS

Hereinafter, the present invention will be described in more detail.

The present invention may provide a composition for shielding radiation comprising poly-gamma-glutamic acid as an active ingredient.

The poly-gamma-glutamic acid may prevent DNA damage induced by irradiation.

The radiation may be gamma rays or neutrons, but it is not limited thereto.

The composition for shielding radiation may contain 0.01 to 1 part by weight of poly-gamma-glutamic acid based on 100 parts by weight of the composition.

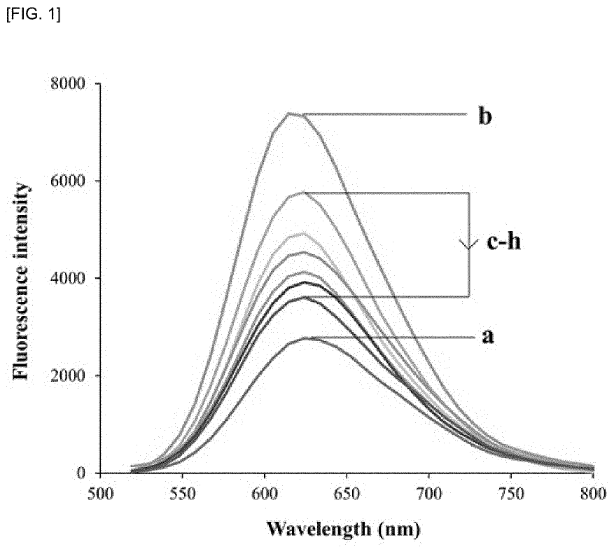

According to an embodiment of the present invention, in order to confirm the radiation shielding effect of poly-gamma-glutamic acid, a gamma ray was irradiated at a dose of 62.6 Gy/min to a total dose of 3756 Gy to natural DNA, and gamma-irradiated DNA was reacted with in ethidium bromide (EtBr) and then measured at a wavelength of 624 nm (excitation at 500 nm, scanning at 510 nm-800 nm) using a fluorescence emission spectrum and as shown in FIG. 1, it was confirmed that the fluorescence intensity was gradually decreased as the irradiation dose was increased as compared with the EtBr-DNA control group and thus that DNA damage was induced by irradiation.

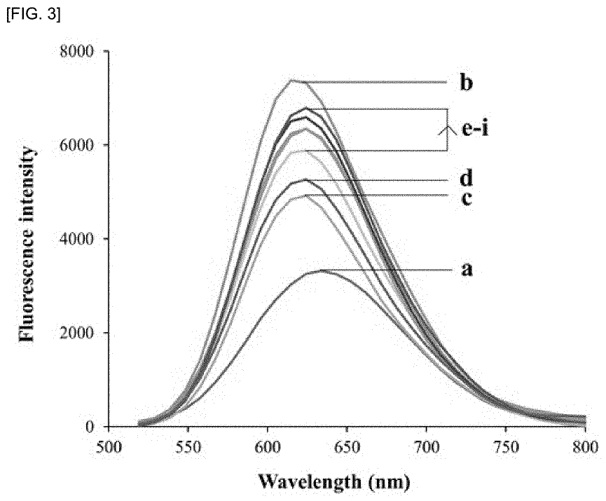

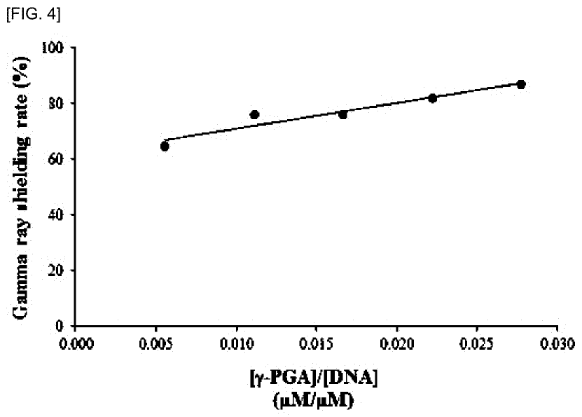

On the other hand, when the DNA to which poly-gamma-glutamic acid (γ-PGA) at a concentration of 0.33 to 1.65 μM was added, was radiated with the radiation having the same conditions as above and as shown in FIG. 3 and FIG. 4, the gamma ray shielding rate of γ-PGA was proved to be 87% at maximum as the concentration of γ-PGA increased, and it was also confirmed that the protection effect of CT-DNA in which the damage was induced by 1252 Gy irradiation of D₅₀ of γ-PGA was excellent and thus the γ-PGA can provide as an excellent a composition for shielding radiation.

Accordingly, the present invention may provide a radiation shielding material comprising poly-gamma-glutamic acid as an active ingredient.

The radiation shielding material may comprise a base layer made of at least one of a mesh, a woven fabric, a nonwoven fabric and a plate; and a radiation shielding layer formed on the surface of the base layer comprising poly-gamma-glutamic acid.

More preferably, the radiation shielding material may be applied to radiation shielding product selected from the group consisting of film for shielding radiation, sheet for shielding radiation, fiber for shielding radiation, coating material for shielding radiation, glass for shielding radiation, building material for shielding radiation, medical tools for shielding radiation and transportation materials for shielding radiation.

The building material for shielding radiation may be selected from the group consisting of concrete, brick, wallpaper and plywood for preventing or blocking radiation transmission, but it is not limited thereto.

The poly-gamma-glutamic acid may be mixed with an object requiring radiation transmission prevention or blocking, placed on the object, coated on the object or immobilized on the object.

In addition, the present invention may provide a method of preparing radiation shielding material comprising: mixing the composition for shielding radiation with an object requiring radiation transmission prevention or blocking, or placing the composition on the object, or coating the composition on the object or immobilizing the composition on the object.

The object may be selected from the group consisting of a film, a sheet, a fiber, a coating material, a glass, a building material, a medical tool and a transportation material.

Hereinafter, the present invention will be described in detail with reference to the following examples. However, the following examples are intended to illustrate the contents of the present invention, but the scope of the present invention is not limited to the following examples. Embodiments of the present invention are provided to more fully describe the present invention to those skilled in the art.

### <EXPERIMENTAL EXAMPLE 1> PREPARATION OF COMPOUND AND EXPERIMENTAL MATERIALS

1. Compound

CT-DNA, a natural DNA which is widely used to bind to anticancer drugs, and D-glutamic acid were purchased from Sigma-Aldrich, USA. EtBr was purchased from aMResco, USA. L-glutamic acid was purchased from SAMCHUN, Korea. Experiments were performed in BPE buffer containing 6 mM NaHPO₄, 2 mM NaH₂PO₄ and 1 mM EDTA at pH 7.0. All reagents are analytical grade, and the third order is used for the experiment. Poly-gamma-glutamic acid was obtained from Bioreaders.

2. Preparation of DNA (Calf thymus DNA, CT-DNA) Solution from Calf Thymus

CT-DNA (20 mg) was dissolved in BPE buffer (6 mM NaHPO₄, 2 mM NaH₂PO₄, 1 mM EDTA, pH 7.0; 10 mL) to obtain a homogeneous DNA solution and stored overnight in a refrigerator to prevent thermal degradation. The DNA concentration expressed as a base pair was measured with a spectrophotometer at 260 nm using a molar extinction coefficient of 6600 M⁻¹cm⁻¹. The DNA stock concentration was 6.86×10⁻³ (M) and the final DNA concentration was about 10⁻⁵ (M).

In addition, the 260/280 ratio of CT-DNA was 1.8, indicating that the DNA is free of protein contamination.

3. Preparation of Ethidium Bromide (EtBr) Solution A stock solution of 10⁻³ (M) EtBr was dissolved in BPE buffer (6 mM NaHPO₄, 2 mM NaH₂PO₄, 1 mM EDTA, pH 7.0) to adjust the final EtBr concentration of 60 μM in order to maximally bind DNA.

4. Preparation of D/L-glutamine Solution

1.36×10⁻¹ (M) D-glutamine and 1.36×10⁻¹ (M) L-glutamate were dissolved in BPE buffer (6 mM NaHPO₄, 2 mM NaH₂PO₄, 1 mM EDTA, pH 7.0) and titrated to pH 6.8. The D/L-glutamine solutions were each mixed in half and titrated to pH 6.8.

5. Preparation of poly-gamma-glutamic Acid Solution

A stock of 3,000 kDa γ-PGA (BioLeaders Corporation, Suji, South Korea) was dissolved in 3.33×10⁻⁶ (M) BPE buffer (6 mM NaHPO₄, 2 mM NaH₂PO₄, 1 mM EDTA, pH 7.0) and titrated to pH 6.8.

### <EXPERIMENTAL EXAMPLE 2> IRRADIATION

Gamma irradiation was performed at a dose rate of 3756 Gy/hr to a total dose of 3756 Gy using a ⁶⁰Co gamma irradiation system (point source AECL, IR-79, MDS Nordion International Co., Ltd., Ottawa, Ontario, Canada) at the Korea Atomic Energy Research Institute (Jeongeup, Korea).

### <EXPERIMENTAL EXAMPLE 3> USE OF FLUORESCENCE SPECTROMETER

The fluorescence emission intensity was measured using an FS-2 fluorescence spectrometer (SCINC), and the radiolysed DNA indicated a decrease in fluorescence intensity of EtBr. When a radiation shielding material is added, the fluorescence intensity increases as compared with the case where there is no radiation shielding material. Two test solutions of a 1.5 ml tube having only CT-DNA without radiation shielding material and a 1.5 ml tube containing radiation shielding materials (GSH and γ-PGA) and CT-DNA were irradiated with gamma radiation and were examined with total doses of 3756 Gy and 1252 Gy, respectively.

In order to confirm the shielding effect of DNA damage by irradiation, 60 μM CT-DNA was irradiated with gamma rays at a dose of 1252 Gy in which 0.33-1.65 μM γ-PGA was added. Both reduced forms of GSH and γ-PGA were dissolved in BPE buffer (6 mM NaHPO₄, 2 mM NaH₂PO₄, 1 mM EDTA, pH 7.0) and fluorescence intensity was measured immediately after irradiation with gamma irradiation.

The fluorophore used in this experiment was ethidium bromide (EtBr) and EtBr was added to the test solution and allowed to react with the CT-DNA so as to form the bonding for 30 min at 37° C. The fluorescence spectrometer measurement conditions were excitation at 500 nm and emission spectra were measured from 510 nm to 800 nm (excitation and emission slit is 10 nm).

### <EXAMPLE 1> IDENTIFICATION OF CT-DNA WITH DAMAGE INDUCED BY IRRADIATION

The CT-DNA solution was reacted at 37° C. for 30 minutes, and then irradiated with gamma rays at a dose of 62.6 Gy/min to a total dose of 3756 Gy. The gamma-irradiated DNA was reacted with EtBr and the fluorescence emission spectrum was measured at a wavelength of 624 nm.

As a result, as shown in FIG. 1, the fluorescence intensity was gradually decreased as the irradiation dose was increased as compared with the EtBr-DNA control group, and the control group was DNA-EtBr exposed to radiation of 0 Gy.

From the above results, it was confirmed that the fluorescence intensity was gradually decreased as the dose of radiation increased.

In addition, the residual amount of double-stranded DNA exposed to radiation was measured by radiation dose of D₅₀ at which 50% of DNA was damaged. The degree of DNA damage induced by radiation was calculated using the formula (I-Iₐ)/(I₀-Iₐ) as the damage rate of DNA (%). At this time, the fluorescence intensity of EtBr was Iₐ, the fluorescence intensity of the EtBr-DNA control was I₀, and the fluorescence intensity of the gamma-irradiated DNA-EtBr was I, and a graph of (I-Iₐ)/(I₀-Iₐ) according to the gamma irradiation was illustrated.

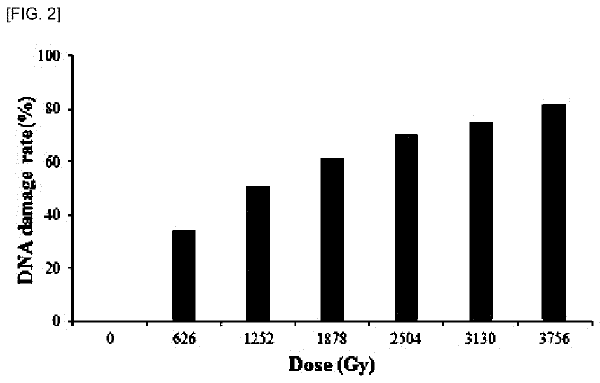

As a result, it was confirmed that a DNA strand break induced by the gamma ray showed a graph which is nearly linear to the dose of gamma ray as shown in FIG. 2, and it was confirmed that a dose of 1252 Gy was D₅₀ (radiation dose at which 50% of DNA was damaged).

From the above results, it was confirmed that the fluorescence intensity of the DNA-EtBr in which damage was induced by irradiation was markedly reduced as compared with the control DNA-EtBr exposed to 0 Gy radiation.

### <EXAMPLE 2> CONFIRMATION OF RADIATION SHIELDING EFFECT OF POLY-GAMMA-GLUTAMIC ACID

To confirm the effect of protecting the DNA from radiation damage, 0.33-1.65 μM of γ-PGA aqueous solution was added to the DNA solution before irradiation.

As a result, the fluorescence intensity of the EtBr-DNA solution was increased as compared with that of the control DNA exposed to 0 Gy as the concentration of γ-PGA increased as shown in FIG. 3 and the D/L-glutamate, a monomer of γ-PGA was confirmed that no radiation shielding effect showed at all.

As shown in FIG. 4, the γ-PGA protection effect from γ-PGA-added DNA damage by gamma-ray irradiation according to the ratio of γ-PGA molar concentration to CT-DNA molar concentration is expressed as gamma ray shielding rate (%) of the protective effect of DNA damage according to increase of γ-PGA concentration. As the amount of γ-PGA gradually increases at 1252 Gy, protection effect of γ-PGA on DNA damage by gamma-ray, i.e. the gamma ray shielding rate was calculated to be 87% at maximum and 87% compared to the control group exposed to 0 Gy.

From the above results, the DNA damage protection effect of γ-PGA was 87%.

In addition, γ-PGA treated with 0.33-1.65 μM concentration showed a protective effect against DNA damage as the treatment concentration increased, and the fluorescence intensity of the EtBr-DNA solution was increased compared to the control DNA exposed to 0 Gy.

Referring to FIG. 3 and FIG. 4, it was confirmed that the protective effect of DNA damage on γ-PGA increased as the concentration of added γ-PGA increased to confirm 87% of the gamma ray shielding rate. As shown in Table 1, the protective effect of CT-DNA with damage induced by 1252 Gy irradiation, D₅₀ of γ-PGA, was shown as the radiation shielding rate of poly-gamma-glutamic acid.

From these results, it was confirmed that γ-PGA has the ability to protect DNA from the damage by shielding radiation.

TABLE 1

| | | | | γ-ray shielding |
|---|---|---|---|---|
| γ-PGA | | DNA | | rate of |
| concentration | | concentration | [γ-PGA]/[DNA] | DNA |
| (μM) | (%) | (μM) | (μM/μM) | (%) |
| 0.33 | 0.1% | 60 | 0.006 | 65 |
| 0.66 | 0.2% | | 0.011 | 76 |
| 0.99 | 0.3% | | 0.017 | 76 |
| 1.33 | 0.4% | | 0.022 | 82 |
| 1.65 | 0.5% | | 0.028 | 87 |

According to the present invention, it was confirmed that the polygamma-glutamic acid polymer which is a biomaterial, exhibits a radiation shielding effect up to 87% at a concentration of 0.33 to 1.65 μM and thus a composition comprising the poly-gamma-glutamic acid is provided as a material for shielding radiation, and a radiation shielding material using the composition and a method of preparing the same are provided.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A method for shielding radiation, comprising:
irradiating gamma rays to a natural DNA;
adding a composition comprising poly-gamma-glutamic acid as an active ingredient; and
measuring a fluorescence intensity of the gamma-irradiated DNA.

2. The method of claim 1, wherein the poly-gamma-glutamic acid prevents DNA damage induced by irradiation.

3. The method of claim 1, wherein the radiation is neutrons.

4. The method of claim 1, wherein the composition for shielding radiation comprises 0.01 to 1 part by weight of the poly-gamma-glutamic acid based on 100 parts by total weight of the composition.

5. A radiation shielding material comprising poly-gamma-glutamic acid as an active ingredient.

6. The radiation shielding material of claim 5, wherein the radiation shielding material is applied to radiation shielding product selected from the group consisting of film for shielding radiation, sheet for shielding radiation, fiber for shielding radiation, coating material for shielding radiation, glass for shielding radiation, building material for shielding radiation, medical tools for shielding radiation and transportation materials for shielding radiation.

7. The radiation shielding material of claim 6, wherein the building material for shielding radiation is selected from the group consisting of concrete, brick, wallpaper and plywood for preventing or blocking radiation transmission.

8. The radiation shielding material of claim 5, wherein the poly-gamma-glutamic acid is mixed with an object requiring radiation transmission prevention or blocking, placed on the object, coated on the object or immobilized on the object.

9. A method of preparing radiation shielding product comprising:
mixing the composition for shielding radiation of claim 1 with an object requiring radiation transmission prevention or blocking, or placing the composition on the object, or coating the composition on the object or immobilizing the composition on the object.

10. The method of preparing radiation shielding product of claim 9, wherein the object is selected from the group consisting of a film, a sheet, a fiber, a coating material, a glass, a building material, a medical tool and a transportation material.
